(19) United States
(12) Patent Application Publication
(10) Pub. No.: US 2024/0252727 A1
(43) Pub. Date: Aug. 1, 2024

(54) Dialysis machine and corresponding method

(71) Applicant: PHYSIDIA, Saint-Barthélémy d'Anjou (FR)

(72) Inventors: Véliana TODOROVA, RILLIEUX LA PAPE (FR); Eric VINCENT, ANGERS (FR)

(21) Appl. No.: 18/560,474

(22) PCT Filed: May 20, 2022
(86) PCT No.: PCT/FR2022/050968

§ 371 Date: Nov. 13, 2023

(30) Foreign Application Priority Data
May 21, 2021 (FR) FR2105337
May 21, 2021 (FR) FR2105339

(51) Int. Cl. A61M 1/16; A61M 39/10; A61M 39/20

(52) U.S. Cl. CPC A61M 1/1621; A61M 39/1011; A61M 39/20; A61M 2205/12; A61M 2205/273

(57) ABSTRACT

A dialysis machine has a connection system (450) for connecting a dialysate supply source (40) to a dialysate delivery system (51). The connection system (450) has a first section (451), which has a conduit (4510) provided at one end with a first connection device (4511) to a first dialysate supply source, and provided at the other end with a second connection device (4512). A second section (452) has a conduit (4520) provided at one end with a connection device (4521) connectable to the second connection device (4512) of the first section (451), in order to allow a series connection of the first section and the second section. The first section (451) is disconnectable from the second section (452) so that, in the disconnected state of the first section (451) from the second section (452), The connection device (4521) of the second section (452) is connectable to a second dialysate supply source.

### FIELD OF THE INVENTION

The present invention relates in general to dialysis machines, in particular for hemodialysis, and also to devices for dialysis machines and to the use (operation and handling) of all or part of such machines.

### PRIOR ART

Dialysis machines are known from the prior art, in particular as described in the application WO2019150058A2.

In the example of the application WO2019150058A2, and as is illustrated in FIG. 1 of the present application, a connection system 450E makes it possible to connect a dialysate supply source to the dialysate delivery system.

The connection system 450E is in the form of a Y-shaped tube, a first branch 451E of which allows the connection to a dialysate supply source for a first dialysis session, and a second branch 452E of which allows the connection to a new dialysate supply source for a second dialysis session. The branches 451E and 452E join at a conduit 455E, forming the foot of the Y, which is in fluidic communication with the dialysate delivery system.

This connection system makes it possible to limit the risk of contamination by allowing one branch to be used for the first session and another branch to be used for the second session, without having to reuse a branch that has already been used.

However, there is a risk that the operator, having disconnected the dialysate supply source from the first branch after a first dialysis session, inadvertently connects the new dialysate supply source to the first branch already used instead of connecting it to the second branch, which has not yet been used. In addition, although the second branch, which has not yet been used, is supposed to be closed by a stopper during the first session, there is a risk of this stopper having been removed inadvertently and of this second branch, which then hangs down without being closed, being contaminated.

It is thus desirable to be able to further reduce the risk of contamination in the dialysis machine during operations performed by the user or by a third party on the machine, in particular when changing the dialysate supply source between two dialysis sessions.

The object of the present invention is to make available a novel dialysis machine and a correspondingly novel method making it possible to overcome all or some of the problems outlined above.

### SUMMARY OF THE INVENTION

To this end, the invention relates to a dialysis machine for treating a body fluid, such as blood or plasma, the dialysis machine comprising:
a dialyzer comprising an enclosure which includes a body fluid passage zone which has a body fluid inlet and a body fluid outlet, and a dialysate passage zone which has a dialysate inlet and a dialysate outlet, and a membrane system between the body fluid passage zone and the dialysate passage zone,
a dialysate delivery system,
a connection system for connecting a dialysate supply source to the dialysate delivery system, the connection system comprising:
a first section, which comprises a conduit provided at one end with a first connection device which is connectable to a connector of a first dialysate supply source, and which is disconnectable from said connector of the first dialysate supply source, and provided at the other end with a second connection device, said conduit being a flexible conduit of constant cross section in the uncrushed state of said conduit, and
a second section, which comprises a conduit which has one end connected to a dialysate delivery system and which is provided at an opposite end with a connection device which is connected, in a disconnectable manner, to the second connection device of the first section, in order to provide a series connection of the first section and the second section, while allowing said first section to be disconnected from the second section, said conduit of the second section being a flexible conduit of constant cross section in the uncrushed state of said conduit, so that, in the state with the first section disconnected and removed from the second section, said connection device of the second section is connectable to a connector of a second dialysate supply source and disconnectable from said connector of the second dialysate supply source.

Thus, the or each other section which is likely to be used subsequently for another dialysis session is not left free waiting for use as in the Y-shaped solution of the prior art, and instead the or each section remains connected in series to the section that is connected to the dialysate supply source in order to allow fluidic communication between the supply source and the dialysate delivery system that passes through said other section or each of said other sections.

The second section is initially disposed between the first section and the dialysate delivery system.

The design of the connection system in the form of two sections (or more when the second section is made up of several sections connected in series) which are connected in series to each other makes it possible to limit the risk of contamination, since the second section does not have an end left free that could trail along and become contaminated. In fact, the ends of each section of the connection system are each connected either to another section, or to the supply source (for the section closest to the supply source) or to the delivery system (for the section closest to the delivery system).

In particular, in the case where there are two sections, the ends of the second section are connected to the first section and to the delivery system. In contrast to the prior art with its Y-shaped connection system, the risk of the user, after a first dialysis session, connecting the supply source to the first section already used is limited, since this first section is intended to be removed.

In addition, the operator understands and visually identifies that the connection system for a subsequent dialysis session must be shorter than for the previous dialysis session, because the previously used section is intended to be removed in order to be able to connect the new supply source to the next section. Thus, the risk of forgetting to remove the section used during the previous dialysis session and of connecting the new dialysate supply source to it for the following dialysis session is reduced.

The machine can also have one or more of the following features taken from any technically permissible combination.

According to one embodiment, the dialysate feed system comprises a dialysate delivery system connected to the feed line, said delivery system comprising at least one flexible bag, called a ventricle bag, intended to contain dialysate, and means for pressurizing the ventricle bag.

According to an optional feature, the device comprises a closing element which, in the closed state, makes it possible to prevent liquid from circulating inside the conduit of the first section.

According to an optional feature, the closing element is of the tamper-evident type so that, in the closed state, the closing element is locked and/or damages the conduit of the first section in order to prevent reuse of the conduit after the closing element has been closed.

According to an optional feature, the second section comprises an activatable and deactivatable opening and closing element making it possible to allow or prevent the circulation of liquid through the conduit of the second section.

According to an optional feature, the first connection device of the first section comprises:
a tubular element capable of being engaged in an opening of the corresponding connector of the first dialysate supply source in order to allow liquid communication inside said tubular element; and
a peripheral wall which extends around and at a distance from the tubular element in order to define an annular space for insertion of a peripheral wall of said connector of the first dialysate supply source.

According to a particular aspect, the first supply source, comprising a bag, a conduit and said connector with which the first connection device of the first section is able to be connected and disconnected, is soldered to the conduit of the first supply source that connects the connector to the bag of the first supply source. In other words, the connector of the first supply source is not removable from the conduit or from the bag of the first supply source.

According to an optional feature, the connection device of the second section comprises:
a tubular element which is capable of being engaged in an opening of the second connection device of the first section in order to allow liquid communication inside said tubular element, and which, in the withdrawn state of the first section, is capable of being engaged in an opening of the connector of the second dialysate supply source in order to allow liquid communication inside said tubular element; and
a peripheral wall which extends around and at a distance from the tubular element in order to define an annular space for insertion of a peripheral wall of said second connection device of the first section or, in the withdrawn state of the first section, to define an annular space for insertion of a peripheral wall of the connector of the second dialysate supply source.

According to an optional feature, the second connection device of the first section comprises a peripheral wall provided with a thread able to cooperate with an internal thread formed in the peripheral wall of the connection device of the second section.

According to an optional feature, the machine comprises:
a dialysate feed line which is connectable to an inlet of a dialysate passage zone of the dialyzer, and
a dialysate discharge line which is connectable to an outlet of the dialysate passage zone of the dialyzer.

According to an optional feature, the discharge line comprises a conduit for connection to a discharge device such as a drainage system and/or a spent dialysate recovery system.

According to an optional feature, the machine comprises at least one flexible bag, called a ventricle bag, intended to contain dialysate and forming part of said delivery system.

According to an optional feature, said at least one bag is included in a device, called a cassette, which is insertable into and removable from a corresponding housing provided in the dialysis machine.

According to an optional feature, the machine comprises a frame and said device, called a cassette, is removable from the frame of the machine.

The invention also relates to a method for successively connecting dialysate supply sources to a connection system of a machine according to any one of the embodiments proposed above, the method comprising the following steps:
providing the connection system in a configuration in which the first section is connected to the second section, the second section being connected by its other end to the dialysate delivery system;
providing a first dialysate supply source having a connector not connected to the connection system;
when a stopper closes the end of the first section opposite the end connected to the second section, removing said stopper;
connecting the first connection device of the first section to the first dialysate supply source, and, after use of the first dialysate supply source:
preferably closing off the first section, for example by closure of a clamp system on the first section,
preferably removing the first dialysate supply source and closing the connection device of the first section with the aid of a stopper;
removing the first section from the second section of the device so that the second section has a free connection device;
providing a second dialysate supply source having a connector not connected to the connection system;
connecting the connector of the second dialysate supply source to the free connection device of said second section.

### BRIEF DESCRIPTION OF THE DRAWINGS

Other features and advantages of the invention will become more clearly apparent from the following description, which is purely illustrative and non-limiting and should be read with reference to the accompanying drawings, in which:

FIG. 1 is a view of a part of a dialysis machine according to an embodiment known from the prior art;

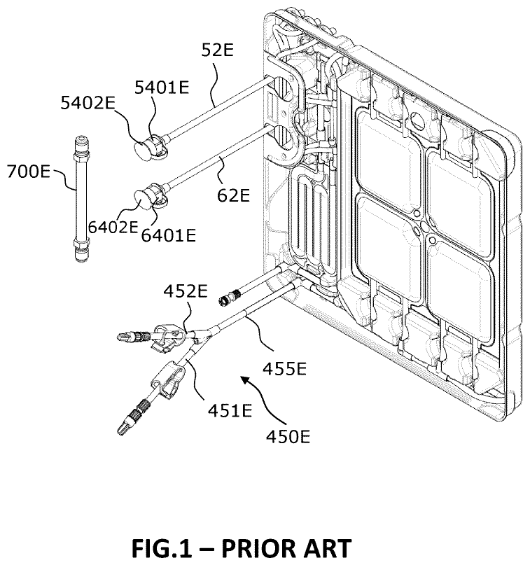

FIG. 2 is a schematic view of a dialysis machine according to an embodiment of the invention, configured for a first dialysis session;

FIG. 3 is a schematic view of a part of the dialysis machine from FIG. 2, during the replacement of dialyzer connection devices by new connection devices intended to be connected to connection devices integral with the dialysate feed and discharge lines, and in the removed state of the previously used dialysate supply source and of the corresponding section of the connection system to the dialysate delivery system;

FIG. 4 is a schematic view of a dialysis machine incorporating elements of the dialysis machine from FIGS. 2 and 3 according to an embodiment of the invention, the machine being configured for a second dialysis session, the dialyzer having been replaced by a new dialyzer connected to the dialysate feed and discharge lines with the aid of new connection devices, and a new dialysate supply source being connected to a remaining section of the connection system to the delivery system;

FIG. 5 is a schematic view of a dialyzer of a dialysis machine according to an embodiment of the invention, during an ultrafiltration step;

FIG. 6 is a schematic view of a dialyzer of a dialysis machine according to an embodiment of the invention, during a backfiltration step;

FIG. 7 is a perspective view of a part of the dialysis machine from FIG. 2, according to an embodiment of the invention, called a cassette, showing each system for connecting the dialysate feed and discharge lines to the dialyzer, in the coupled state of the second connection device to the first connection device and in the closed state of the stopper of the second connection device, and showing a connection system for connecting a dialysate supply source to a dialysate delivery system for a first dialysis session;

FIG. 8 is a view of each dialyzer connection system from FIG. 7 in the open state of the stopper of the second connector of the second connection device of each connection system;

FIG. 9 is a view of the connection system of the dialysate supply source configured for a first dialysis session;

FIG. 10 is a view of each dialyzer connection system from FIG. 8 in the state in which the second connection device is disconnected from the first connection device of each connection system;

FIG. 11 is a view of the connection system of the dialysate supply source, in the disconnected state of the first section of the system in order to allow a new dialysate supply source to be connected to the free end of the second section of the system for a second dialysis session;

FIG. 12 is a view showing the first connection device of each connection system from FIG. 8, which remains fixed to the corresponding feed or discharge line, and a new second connection device, identical to the second connection device that has been removed, and of which the first connector, which is connectable to the first connection device, is provided with a stopper, which is shown in the open state;

FIG. 13 is a view of the dialyzer connection systems from FIG. 12, in the connected state, for each connection system, of the first connection device with the new second connection device, and, in the open state of the stopper, of the second connector of the second connection device of each connection system;

FIG. 14 is a view of the connection system of the dialysate supply source, in the state with a new dialysate supply source connected to the second remaining section of the connection system for a second dialysis session;

FIG. 15 is a perspective view of a dialysate supply source housed in a support structure and connectable to the cassette of the dialysis machine according to an embodiment of the invention;

FIG. 16 is a diagram showing several steps of a method for replacing a dialyzer and a dialysate supply source according to an embodiment of the invention.

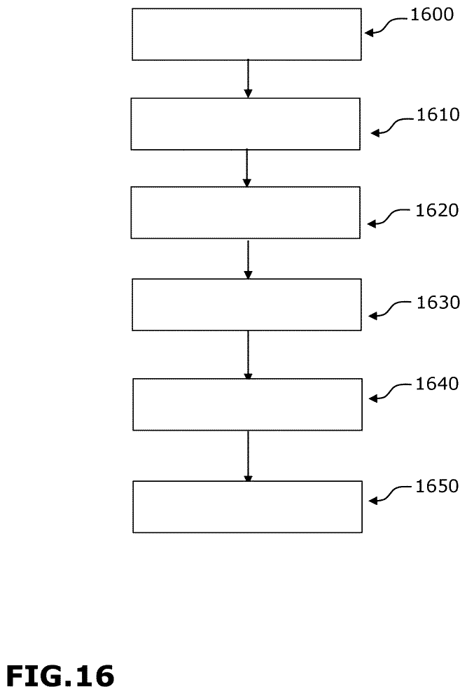

### DETAILED DESCRIPTION

The concept of the invention is described more completely below with reference to the accompanying drawings, in which embodiments of the concept of the invention are shown. In the drawings, the size and relative scale of the elements may be exaggerated for the sake of clarity. Similar numbers refer to similar elements across all of the drawings. However, this concept of the invention can be implemented in many different forms and should not be interpreted as being limited to the embodiments set forth here. Instead, these embodiments are proposed so that this description is complete and communicates the scope of the concept of the invention to those skilled in the art.

Throughout the specification, a reference to “an embodiment” means that a functionality, a structure or a particular feature described in connection with an embodiment is included in at least one embodiment of the present invention. Thus, where the expression “in an embodiment” appears at various points throughout the specification, this does not necessarily refer to the same embodiment. Furthermore, the functionalities, structures or particular features may be combined in any appropriate way in one or more embodiments.

### GENERAL ASPECTS

As has been set out above, the invention relates to a device for a dialysis machine, a corresponding dialysis machine and a corresponding method of use making it possible to treat a body fluid, such as blood or plasma, with a reduced risk of contamination when an operator, who may be the user or a third party, has to work on the machine, in particular when changing the dialyzer (presented below) between two dialysis sessions, and/or when changing the dialysate supply source (presented below) between two dialysis sessions.

The term “body fluid” is understood to mean a fluid of the type present in the human or animal body, such as blood or plasma.

The dialysis machine is used to treat a body fluid, such as blood or plasma. The term “body fluid” is understood to mean a fluid of the type present in the human or animal body, such as blood or plasma. In the remainder of the description, the body fluid in question is blood, but it could of course be another body fluid such as plasma.

According to one embodiment, the dialysis machine can incorporate the elements described in the international application number WO2013050689A1 or WO2019150058A2. However, the dialysis machine according to the invention differs from those described in said international applications WO2013050689A1 and WO2019150058A2 at least in terms of the system for connecting the dialysate supply source to the dialysate delivery system as explained below, and preferably also in terms of the system for connecting the dialysate feed line to the dialyzer inlet and/or in terms of the system for connecting the dialysate discharge line to the dialyzer outlet, as explained below.

### Dialyzer

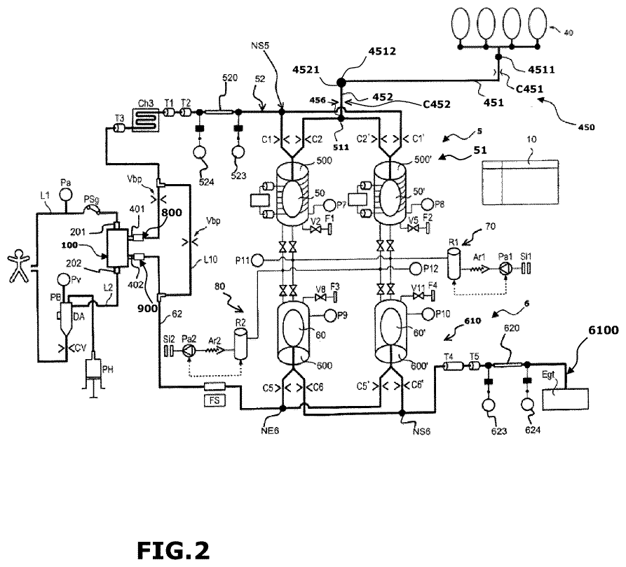

The machine comprises a dialyzer 100. The dialyzer is also usually called a dialyzer filter. As is illustrated in FIGS. 2, 5 and 6, the dialyzer 100 is in the form of an enclosure which includes a body fluid passage zone 12, called the blood compartment, which has an inlet 201 for body fluid and an outlet 202 for body fluid. The chamber also includes a dialysate passage zone 14, also called the dialysate compartment, which has a dialysate inlet 401 and a dialysate outlet 402.

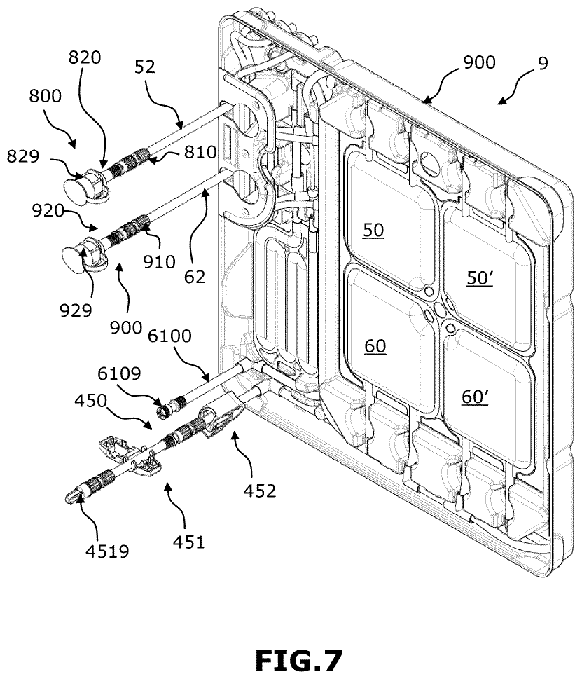

The dialysate inlet 401 and the dialysate outlet 402 are intended to be connected by connection systems 800, 900 (presented below) to a dialysate feed line 52 and a dialysate discharge line 62, respectively (see FIG. 7).

Dialysate is a liquid well known to those skilled in the art, its composition being close to physiological saline. Dialysate includes water of sufficient mineral and bacteriological quality. This water is added to a concentrate, in the form of a powder of mineral salts or in the form of a liquid concentrate, to make the required dialysate.

Physiological saline is usually composed of a mixture of water and sodium chloride. Dialysate is generally composed of physiological saline to which glucose and ions such as potassium, magnesium, calcium and optionally lactate and/or bicarbonate are added according to the needs of the patient.

According to a preferred aspect, the dialysate used is non-pyrogenic sterile dialysate. The dialysate has been sterilized, for example, in an autoclave. This dialysate can be made from purified water and salt.

The dialysate circulates through the dialysate compartment 14, generally in the opposite direction to the blood circulation in the blood compartment 12. The dialysate generally circulates in an open circuit. It is also possible to provide a loop for partial regeneration of clean dialysate from the dialysate charged with toxins.

### Membrane

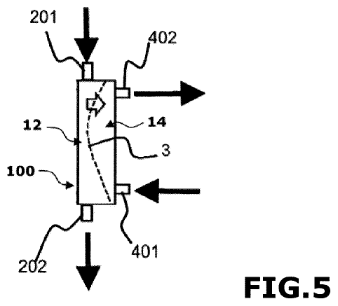

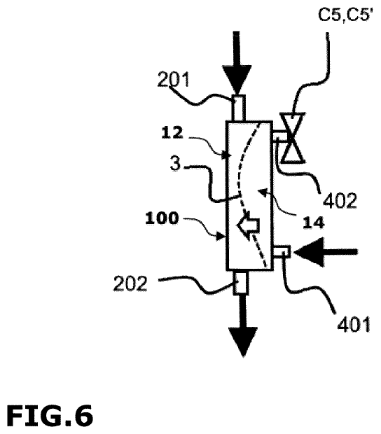

As is illustrated in FIGS. 5 and 6, the enclosure also includes a membrane system 3 to allow an exchange of elements (substances, molecules) between the body fluid present in the body fluid passage zone 12 and the dialysate present in the dialysate passage zone 14.

From a functional point of view, the dialyzer 100 can be schematized in the form of an enclosure accommodating a dialysis membrane 3 which separates the chamber into a blood compartment, corresponding to the body fluid passage zone 12, and a dialysate compartment, corresponding to the dialysate passage zone 14.

The semipermeable dialysis membrane system 3 is designed to allow a fraction of the blood volume to pass through when the difference between the local pressure in the blood compartment 12 and the local pressure in the dialysate compartment 14 is greater than a given value. This pressure difference is called the transmembrane pressure. In particular, the membrane system is designed in such a way that only the smallest molecules can pass through the holes or pores of the membrane system, namely water, mineral salts, and molecules of small to medium molecular weight.

More specifically, the membrane system 3 can comprise a membrane designed such that, when the difference between the pressure exerted on at least a portion of the membrane on the side of the blood compartment 12 and the pressure exerted on said portion of the membrane on the side of the dialysate compartment 14 is greater than a given threshold value, said portion of the membrane allows an aqueous fraction of the blood to pass into the dialysate compartment 14, according to a convective phenomenon called ultrafiltration. Conversely, said membrane 3 is designed in such a way that, when the difference between the pressure exerted on said portion of the membrane 3 on the side of the dialysate compartment 14 and the pressure exerted on said portion of the membrane on the side of the blood compartment 12 is greater than a given threshold value, said portion of the membrane allows the dialysate to pass into the blood compartment 12, according to a phenomenon called backfiltration.

The dialysis membrane system can be in the form of capillary fibers, with the blood circulating inside the fibers, the dialysate outside.

The blood inlet 201 is connected to an arterial line L1, one end of which is connected to a fistula of the patient so as to be able to extract the blood from the body of the patient to be treated in order to carry out a dialysis session. In addition, the blood outlet 202 is connected to a venous line L2, one end of which is connected to a vein of the patient in order to reintroduce the blood into the body of the patient after treatment. The term “line” is understood to mean a tube which possibly comprises several branches or portions and through which a liquid can circulate. According to a particular aspect, these lines can be linked to each other so that one cannot be removed without the other.

In the example shown in FIG. 2, the arterial line L1 is also equipped with a blood pump Psg and with a pressure sensor Pa, also known as an arterial pressure sensor. The venous line L2 comprises a pressure sensor Pv, also called a venous pressure sensor, a bubble trap PB, and an air detector DA. Advantageously, the pressure sensor Pv is connected to the bubble trap PB. Preferably, said venous line L2 also comprises an anticoagulant injection system, such as a heparin pump PH.

The arterial line L1, the venous line L2 and the blood compartment 12 form the blood circuit of the machine.

### Dialysate Circuit

As is illustrated in FIG. 2, the machine also comprises a dialysate feed system 5, which comprises a dialysate feed line 52 which is connected to the inlet 401 of the dialysate compartment 14, and a dialysate discharge system 6, which comprises a discharge line 62 connected to the outlet 402 of the dialysate compartment 14.

Said dialysate feed system 5, the dialysate compartment 14, and said dialysate discharge system 6 form the dialysate circuit.

As has been set out above, the dialysate feed line 52 is connected to the inlet 401 of the dialysate passage zone 14 by a connection system 800.

### Feed Line Connection System 800

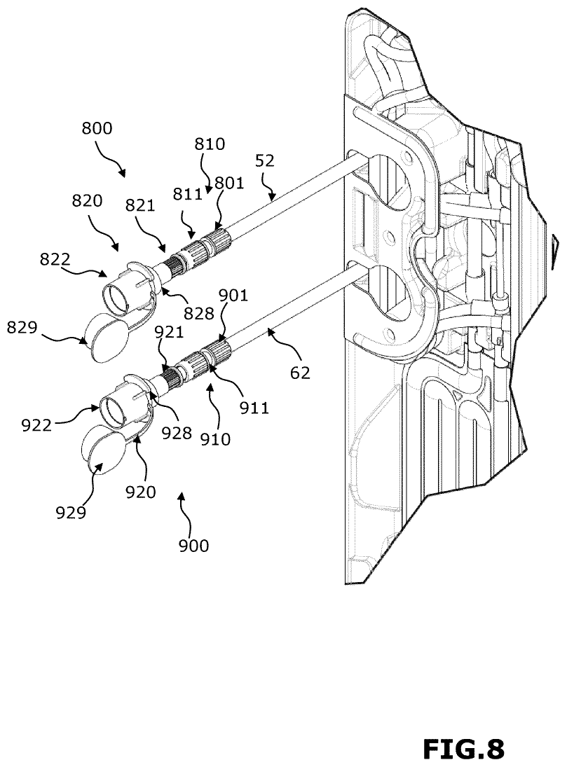

As is illustrated more particularly in FIG. 8, the connection system 800 comprises a first connection device 810 fixed, for example by gluing, to the feed line 52, and a second connection device 820 connectable to the first connection device 810, to allow fluidic communication through the first connection device and the second connection device. The second connection device 820 can be disconnected from the first connection device 810.

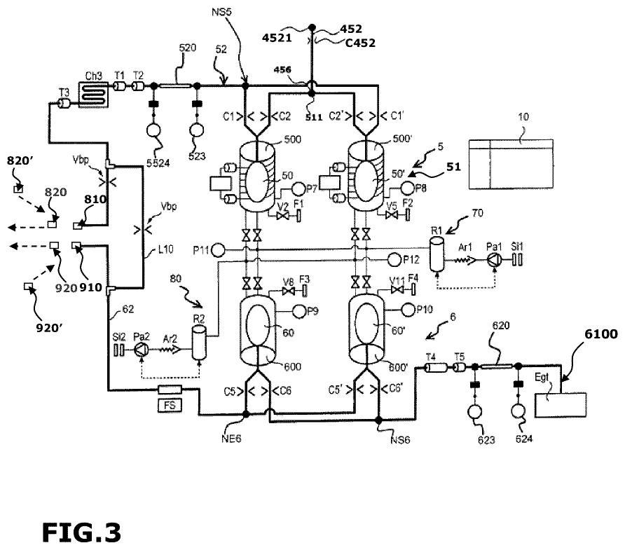

The second connection device 820, connected to the first connection device 810, can thus be connected to the dialysate inlet 401 of the dialyzer 100 for a first dialysis session, then be disconnected from the first connection device 810 which remains fixed on the feed line, for example at the same time as the dialyzer is withdrawn (or separately), in order to be able to replace this second connection device 820 by a new second connection device 820′, as is illustrated in FIGS. 3, 4, 12 and 13.

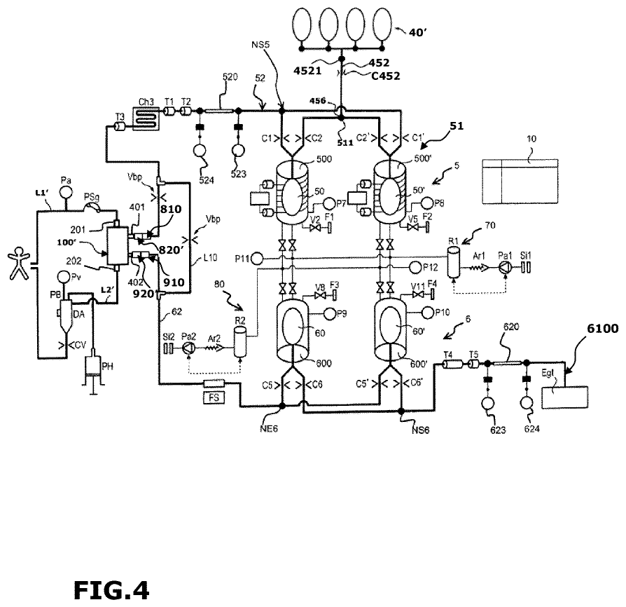

This new second connection device 820′, considered to be clean or sterile, can then be connected by one end to the first connection device 810 and by its other end to the dialysate inlet 401 of a new dialyzer 100′, as is illustrated in FIG. 4.

In the embodiment illustrated in FIG. 8, the second connection device 820, which is removably connectable to the first connection device 810, comprises a female Hansen connector 822 and a female Luer Lock connector 821. These two connectors 821, 822 are connected to each other by a tube. According to a particular aspect, the Hansen connectors correspond to the NF EN ISO 8637 standard. Preferably, the first connection device 810 fixed to the feed line 52 comprises a male Luer Lock connector 811 connectable to the connector 821, and a female Luer Lock connector 801 at the other side connected to the line 52. According to a particular aspect, the Luer Lock connectors correspond to the NF EN ISO 594 standard.

The connector 811 comprises a tubular element 812 capable of being engaged in the opening of the first connector 821 of the second connection device 820, in order to be in fluidic communication with the opening of the first connector 821 of the second connection device 820, and a peripheral wall 813 which extends around and at a distance from the tubular element 812 in order to define an annular space for insertion of the peripheral wall of the first connector 821 of the second connection device 820 described below. Advantageously, the peripheral wall 813 is internally threaded in order to cooperate with a thread 821a of the first connector 821 formed on at least part of the external face of its peripheral wall.

The first connector 821 of the second connection device 820 is in the form of a tubular element, preferably straight, which defines an opening emerging at one end of said second connection device 820 in order to receive said tubular element 812 of the connector 811. The first connector 821 is thus able to cooperate with the connector 811 of the first connection device 810 by engagement of the peripheral wall of the first connector 821 between the tubular element 812 and the peripheral wall 813 of the connector 811 of the first connection device 810.

The second connector 822 of the second connection device 820, opposite the first connector 821, can be in the form of a tubular element of circular cross section, preferably with a diameter greater than that of the first connector 821. The second connector 822 is connectable to the dialysate inlet 401 of the dialyzer.

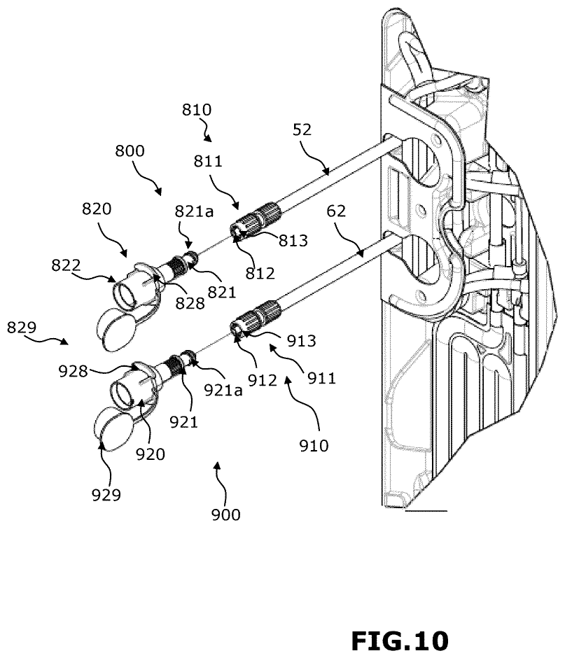

According to one embodiment, and as is illustrated in FIG. 10, the second connection device 820 has lateral fins 828 making it easier to grasp the second connection device 820 in order to disconnect it.

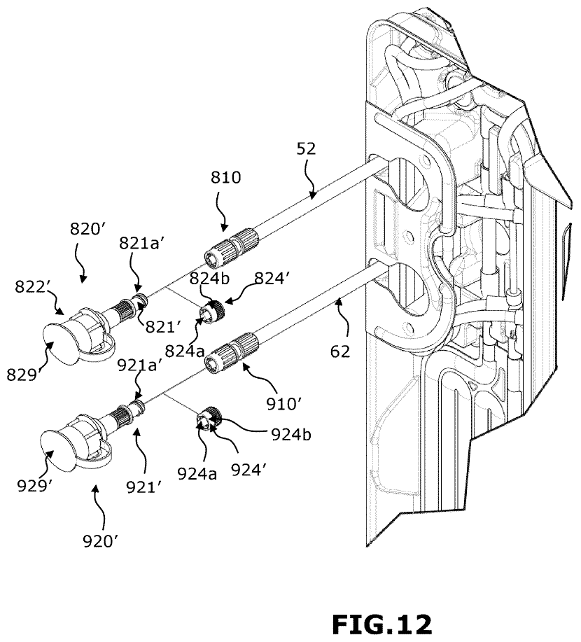

In the state in which the second connection device 820 is not yet connected to the first connection device 810, the second connection device 820 is fitted with a stopper making it possible to close the opening of the first connector 821 of the second connection device 820, while being removable in order to free the opening of the first connector 821 of the second connection device 820 and to be able to connect the second connection device 820 to the first connection device 810. Such a stopper is shown and labeled 824′ in FIG. 12 for the second connection device 820′.

The stopper 824′ comprises a stud 824a capable of being engaged in the opening of the first connector 821′ of the second connection device 820′, and a peripheral wall 824b which extends around and at a distance from the stud 824a in order to define an annular space for insertion of the peripheral wall of the first connector 821′. The peripheral wall 824b is preferably internally threaded in order to cooperate with the thread 821a′.

The second connector 822 of the second connection device 820, intended to be connected to the inlet 401 of the dialyzer 100, is provided with a stopper 829 making it possible to close the opening of the second connector 822, while being removable in order to free the opening of the second connector 822. Advantageously, the stopper 829 remains attached to the second connector 822 by an attachment element such as a wire, so as to prevent it from falling off or being lost.

As is illustrated in FIGS. 3, 4, 12 and 13, the second connection device 820 can be withdrawn and replaced by a new second connection device 820′, identical to the one withdrawn. This limits the risk of contamination when changing the dialyzer at the end of the feed line intended to be connected to the dialyzer, while having to change only a part of the connection system which is likely to have been contaminated at the level of the stopper which has remained open. In particular, such a system makes it possible to limit the risk of bacteriological contamination/proliferation, during a period of in which the machine is not in use (or is on standby) between a first dialysis session and a second dialysis session, since the new second connection device 820′ can be connected and left in the closed position of the stopper 829 during the period in which the machine is not in use (or is on standby) between the first and second dialysis sessions.

The construction of the connection system 800 in two parts (as also for the system 900 presented below), with a fixed part which remains on the feed line and with a part which is connectable to the dialyzer and can be disconnected from the fixed part, allows said part which is connectable to the dialyzer, and which is likely to be contaminated, to be replaced by a new sterile identical part, thus limiting the risk of contamination during replacement of the dialyzer.

### Dialysate Delivery System 51

The feed system 5 also comprises a dialysate delivery system 51 connected to the feed line 52. Said dialysate feed system 5 makes it possible to deliver dialysate into the dialysate compartment 14 via the feed line 52.

According to one embodiment, said delivery system 51 comprises at least one flexible bag 50, called a ventricle bag, intended to contain dialysate. Said system 51 can comprise several bags, as in the example illustrated in the figures.

Preferably, said dialysate feed system 5 comprises an additional ventricle bag 50′ which is mounted to bypass the portion of the feed line 52 to which said ventricle bag 50 is connected.

In the remainder of the description, reference is made to a system 51 comprising several bags 50, 50′, but it will be understood that this description also applies to the case of a delivery system 51 comprising a single bag. Each ventricle bag of the system 51 is intended to contain dialysate and is connected to said feed line 52.

Each ventricle bag 50, 50′ is housed in a substantially sealed enclosure 500, 500′ which can be placed under pressure, and possibly under vacuum. Each ventricle bag 50, 50′ can thus be placed under pressure by pressurizing means 70 and, optionally, under vacuum by a vacuum generator 80.

Said pressurizing means 70 comprise a device for injection of pressurized gas, such as an air compressor or a cylinder of compressed gas, capable of injecting pressurized gas into the sealed enclosure 500, 500′ in which the ventricle bag 50, 50′ is housed. Said enclosure has an outlet, the opening of which is controllable by a solenoid valve in order to reduce the pressure in the enclosure.

The application of a pressure to the ventricle bag 50 or 50′ and the regulation of this applied pressure, with the aid of the pressure difference measured across the pressure drop tube 520, make it possible to maintain, for a given time, a fixed flow rate of dialysate in the dialysate compartment in a reliable and precise manner, and thus to control backfiltration operations. The application and the regulation of the pressure applied to the ventricle bag 50 or 50′ also make it possible, by adjusting the pressure applied to the discharge bag 60 or 60′, to maintain, for a given time, a given average pressure in the dialysate compartment, and thus to control ultrafiltration operations.

The two ventricle bags 50, 50′ are able to be placed under pressure/vacuum independently of each other.

### System for Opening/Closing the Feed Line

As is illustrated for example in FIG. 2, each ventricle bag 50, 50′ is also provided with a downstream closing/opening member C1, C1′ for the feed line which, in the open state, permits flow of dialysate from the corresponding bag 50, 50′ to the inlet 401 of the dialysate compartment, under the effect of a pressure applied to said ventricle bag by the corresponding means 70, and, in the closed state, prevents said flow of dialysate.

Each downstream closing/opening member C1, C1′ of the feed line is situated between the corresponding bag 50, 50′ and the outlet node NS5 for connecting the two bags 50, 50′. Each ventricle bag 50, 50′ is also provided with an upstream closing/opening member C2, C2′ which permits or prevents the supply to said ventricle bag 50, 50′ from a supply source 40. The supply source 40 can comprise a plurality of feeder bags.

A connection system 450 for connecting a dialysate supply source to the dialysate delivery system 51 is described below.

In the example illustrated in FIGS. 2 to 4, each portion of the dialysate feed line 52 at which an upstream C2, C2′ or downstream C1, C1′ closing/opening member is located is flexible. Each of said closing/opening members C2, C2′, C1, C1′ is formed by a controllable clamp for, on the one hand, clamping the wall of said flexible portion of the feed line 52 from the outside so as to close this portion, and, on the other hand, for leaving open said flexible portion of said feed line 52.

The presence of the two ventricle bags and of the alternating clamp system formed by the members C1 to C2′ makes it possible to successively and alternately load the ventricle bags from the supply source 40. This makes it possible to continuously supply the dialysate feed line from one of the ventricle bags.

Placing the enclosure of a ventricle bag 50 or 50′ under vacuum with the aid of the vacuum generator 80 makes it possible, in the open state of the upstream opening/closing member C2 or C2′, and in the closed state of the corresponding downstream opening/closing member C1 or C1′, to aspirate the dialysate contained in the supply source 40 or 40′ in order to fill said ventricle bag 50 or 50′.

To control the flow of dialysate in the feed line 52, the control unit 10 controls the opening of the downstream member C1 or C1′ of one of the ventricle bags 50 or 50′ and the closure of the corresponding upstream member C2 or C2′. The use of two ventricle bags mounted in parallel allows one to be recharged while the other is in use.

According to one embodiment, the dialysate feed line 52 comprises a passage restriction 520 in order to create a pressure drop and to allow the pressure applied to the ventricle bag 50 or 50′ to be regulated as a function of the pressure difference measured across said restriction. In particular, said line 52 is provided with means of measuring the pressure difference between the inlet and the outlet of said passage restriction 520. This restriction 520 can be formed at least in part by a calibrated tube of constant cross section which forms a narrowing over a predetermined length. Said passage restriction 520 is situated between the bags 50, 50′ and the inlet 401 of the dialysate compartment 14.

The connection NS5 of the delivery system 51 to said feed line 52 is situated upstream of the passage restriction 520.

Said pressurizing means advantageously maintain a pressure on the ventricle bag 50 or 50′ that is sufficient to cause the flow of the dialysate. The feed line is calibrated such that, for a determined pressure applied to the ventricle bag 50 or 50′, the flow of dialysate in said line has a substantially constant value.

Such a system formed by a pressure drop tube and by means for measuring the pressure difference across the restriction makes it possible in particular to determine the flow rate of dialysate which circulates in said feed line and which enters the dialysate compartment. By virtue of the dialysate flow rate thus measured, the control unit 10 can regulate the pressure applied to the ventricle bag 50 or 50′ in order to obtain the desired flow rate of dialysate entering the dialysate compartment and to precisely and reliably maintain this flow rate at a given value for a given time.

Said means for measuring the pressure difference across the restriction 520 of the feed line 52 can comprise two pressure tap orifices formed in the peripheral wall of said line 52, one upstream and the other downstream of said restriction 520. Each pressure tap orifice is associated with a pressure sensor 523, 524. Each pressure sensor 523, 524 is arranged relative to the corresponding pressure tap orifice in such a way as to measure the pressure in the feed line 52 at the level of said pressure tap orifice while being distanced from said orifice in order not to be in contact with the dialysate circulating in the line 52, 62.

The pressure sensors 523, 524 can be configured as described in the international application WO2013050689.

Provision can be made that the pressure measurement in the arterial line L1 and the venous line L2 is carried out in the same way as for the pressure measurement across the restriction 520. It is thus possible to provide that the pressure sensor Pa and the pressure sensor Pv are fixed in a part of the frame at a distance from a pressure orifice formed in the corresponding line L1, L2. According to a particular aspect, the pressure sensors Pa and Pv measure the pressure in the corresponding line L1, L2 without risk of contamination.

### Dialysate Discharge System

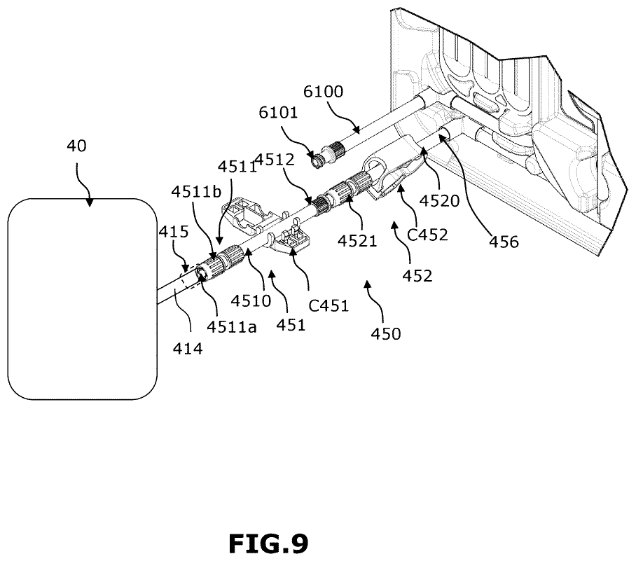

The dialysate discharge system 6 comprises a dialysate discharge line 62 which is connected to the outlet 402 of the dialysate compartment 14, as set out in detail below. In the example illustrated more particularly in FIGS. 7 and 9, the dialysate discharge system 6 also comprises a conduit 6100 intended to be connected, for example at the level of the endpiece 6101 (which can be closed by a stopper 6109), to a recovery container or to a drainage system Egt.

### Connection System 900

As is illustrated more particularly in FIG. 8, and in a manner similar to the connection system 800, the connection system 900 comprises a first connection device 910 fixed to the discharge line 62, and a second connection device 920 connectable to the first connection device 910, to allow fluidic communication through the first connection device and the second connection device. The second connection device 920 can be disconnected from the first connection device 910.

The second connection device 920, connected to the first connection device 910, can thus be connected to the dialysate outlet 402 of the dialyzer 100 for a first dialysis session, and then be disconnected from the first connection device 910, for example, at the same time as the dialyzer is withdrawn (or separately), in order to be able to replace this second connection device 920 by a new second connection device 920′.

This new second connection device 920′, considered to be clean or sterile, can then be connected by one end to the first connection device 910 and by its other end to the dialysate outlet 402 of the new dialyzer 100′.

In the embodiment illustrated in FIG. 8, the second connection device 920, which is removably connectable to the first connection device 910, comprises a female Hansen connector 922 and a female Luer Lock connector 921. These two connectors 921, 922 are connected to each other by a tube. According to a particular aspect, the Hansen connectors correspond to the NF EN ISO 8637 standard. Preferably, the first connection device 910 fixed to the discharge line 62 comprises a male Luer Lock connector 911 connectable to the connector 921, and a female Luer Lock connector 901 at the other side connected to the line 52. According to a particular aspect, the Luer Lock connectors correspond to the NF EN ISO 594 standard.

In the example illustrated in FIGS. 2 to 15, the connection system 900 comprises elements identical to those of the connection system 800 presented above.

The connector 911 comprises a tubular element 912 capable of being engaged in the opening of the first connector 921 of the second connection device 920, in order to be in fluidic communication with the opening of the first connector 921 of the second connection device 920, and a peripheral wall 913 which extends around and at a distance from the tubular element 912 in order to define an annular space for insertion of the peripheral wall of the first connector 921 of the second connection device 920 described below.

Preferably, and as is illustrated in FIG. 10, said first connector 921 is provided with a thread 921a, on at least a part of the external face of its peripheral wall, capable of cooperating with an internal thread formed inside the wall 913.

The first connector 921 is able to cooperate with the connector 911 of the first connection device 910 by engagement of the peripheral wall of the first connector 921 between the tubular element 912 and the peripheral wall 913 of the connector 911.

The second connector 922 of the second connection device 920, opposite the first connector 921, can be in the form of a tubular element of circular cross section, preferably with a diameter greater than that of the first connector 921. The second connector 922 is connectable to the dialysate outlet 402 of the dialyzer.

According to one embodiment, and as is illustrated in FIG. 10, the second connection device 920 has lateral fins 928 making it easier to grasp the second connection device 920 in order to remove it.

In the state in which the second connection device 920 is not yet connected to the first connection device 910, the second connection device 920 comprises a stopper making it possible to close the opening of the first connector 921 of the second connection device 920, while being removable in order to free the opening of the first connector 921 of the second connection device 920. This stopper is shown and labeled 924′ in FIG. 12 for the second connection device 920′.

The stopper 924′ comprises a stud 924a capable of being engaged in the opening of the first connector 921′ of the second connection device 920′, and a peripheral wall 924b which extends around and at a distance from the stud 924a in order to define an annular space for insertion of the peripheral wall of the first connector 921′ and which is preferably internally threaded in order to cooperate with the thread 921a.

The second connector 922 of the second connection device 920 is provided with the stopper 929 making it possible to close the opening of the second connector 922, while being removable in order to free the opening of the second connector 922. Advantageously, the stopper 929 is attached to the second connector 922 by an attachment element, such as a wire, so as to prevent it from falling off or being lost.

The stopper 929′ of the second connection device 920′ can be left closed during the period of stoppage or standby between two dialysis sessions, before the connection of the new dialyzer 100′.

Similarly to the connection system 800, the connection system 900 makes it possible to limit the risk of bacteriological contamination/proliferation at the level of the corresponding line 62, between a first session and a second session.

### System for Opening/Closing the Discharge Line

Said machine comprises a system, preferably of the clamp type, for opening/closing the dialysate discharge line 62. The opening/closing system, in the open state, permits the circulation in said discharge line 62 of the dialysate present at the outlet 402 of the dialysate compartment 14 for filling one of the discharge bags 60 or 60′, and, in the closed state, prevents the circulation of the dialysate in said discharge line 62 such that, for a sufficient pressure in the dialysate compartment, said dialysate passes through the membrane 3 into the blood compartment.

In the example illustrated in FIG. 2, said dialysate discharge system 6 comprises at least one discharge bag 60, preferably flexible, having an inlet and an outlet that are connected to the dialysate discharge line 62. Said opening/closing system comprises an upstream opening/closing member C5 situated between the outlet 402 of the dialysate compartment 14 and said discharge bag 60. A closing/opening member can also be provided on the line 62.

Advantageously, said dialysate discharge system 6 comprises at least one other discharge bag 60′ mounted to bypass said discharge bag 60. Thus, the discharge bag 60 and the discharge bag 60′ are situated on two branches of the discharge line 62. In other words, starting from the outlet of the dialysate compartment 14, the discharge line 62 separates, upstream of the discharge bags 60, 60′, into two parallel branches, one provided for the discharge bag 60, the other for the discharge bag 60′. These two branches join each other downstream of the bags 60, 60′ and upstream of the cross-sectional restriction 620, as is set out in detail below.

Said opening/closing system comprises another upstream opening/closing member C5′ situated between said other discharge bag 60′ and the entry node NE6 for connecting the discharge bags 60, 60′.

Similarly to the upstream members C5, C5′, each discharge bag 60, 60′ is provided with a downstream closing/opening member C6, C6′ situated between the corresponding discharge bag 60, 60′ and the outlet node NS6 for connecting the discharge bags 60, 60′.

Each portion of the dialysate discharge line 62 at which an upstream C5, C5′ or downstream C6, C6′ closing/opening member is situated is flexible. Each of said upstream C5, C5′ or downstream C6, C6′ members for closing/opening the dialysate discharge line 62 is formed by a controllable clamp for, on the one hand, clamping the wall of said flexible portion of the discharge line 62 from the outside, so as to close this portion, and, on the other hand, for leaving open said flexible portion of said discharge line 62.

Such a set of upstream C5, C5′ and downstream C6, C6′ closing/opening members, associated with the discharge bags 60, 60′, makes it possible, in the open state of the upstream member C5, respectively C5′, and in the closed state of the downstream member C6, respectively C6′, to fill the discharge bag 60, respectively 60′, and, in the closed state of the upstream member C5, respectively C5′, and in the open state of the downstream member C6, respectively C6′, to empty said discharge bag 60, respectively 60′.

In the case where the discharge system 6 is formed by one or more discharge bags 60, 60′, the discharge line 62 is considered open when the dialysate is able to flow through said line in order to fill said bag or one of said bags.

The means for discharging the dialysate and the liquid present in the discharge bag 60 or 60′ can be formed by gravity if the configuration of the discharge bag lends itself to this and/or by pressurizing means 70, preferably common to those that are used to pressurize the ventricle bags 50, 50′. In the latter case, said or each bag 60, 60′ is housed in a substantially sealed enclosure 600, 600′ capable of being pressurized by said pressurizing means 70. Said enclosure has an outlet, the opening of which is controllable by a solenoid valve in order to reduce the pressure in the enclosure.

Provision can also be made that each enclosure 600, 600′ which houses a discharge bag 60, 60′ is connected to a vacuum generator 80. The vacuum generator 80 and/or the pressurizing means 70 make it possible to apply a pressure or a vacuum to the discharge bag 60 or 60′, so as to adjust the average pressure in the dialysate compartment 14, for example during an ultrafiltration phase. In this case, the downstream member C6 or C6′ of the discharge bag 60 or 60′ which is used to adjust the average pressure in the dialysate compartment 14 is closed while the corresponding upstream member C5 or C5′ is open.

Said vacuum generator 80 may be common for placing the enclosures of the dialysate feed system 5 under vacuum and for placing the enclosures of the discharge system 6 under vacuum.

Said machine comprises means for determining the quantity of dialysate and liquid recovered in the discharge bag 60 or 60′ which empties.

According to one embodiment, said means for determining the quantity of dialysate and liquid recovered in the discharge bag(s) 60, 60′ comprise a passage restriction 620, formed in the dialysate discharge line 62 and situated downstream of said discharge bags 60, 60′, and means for measuring the pressure difference across said passage restriction 620.

Said means for measuring the pressure difference across the restriction 620 of the discharge line 62 may be similar to those associated with the restriction 520 of the feed line 52.

Thus, said means for measuring the pressure difference across the restriction 620 of the discharge line 62 can comprise two pressure tap orifices formed in the peripheral wall of said line 62, one upstream and the other downstream of said restriction 620. According to a particular aspect, each pressure tap orifice is associated with a pressure sensor arranged so as to measure the pressure in the line 62 at said orifice while being distanced from said orifice so as not to be in contact with the dialysate circulating in the line 62.

In a manner similar to the means for measuring the pressure difference across the restriction 520, a filter permeable to air and impermeable to infectious agents and to liquids can be interposed between each pressure sensor 623, 624 and the corresponding pressure tap orifice. In particular, each pressure sensor 623, 624 is mounted in a cavity of the frame of the machine with the aid of a hollow support piece which is intended to be coupled to the conduit 621, 622 connected to the corresponding pressure tap orifice.

The pressure sensors 623, 624 associated with said restriction 620 make it possible to determine the pressure difference across the restriction 620 and thus the flow rate of dialysate and of liquid discharged from the discharge bag 60 or 60′, which makes it possible to determine, by measuring the corresponding flow time, the quantity of dialysate and of liquid recovered in said discharge bag 60 or 60′.

### Supply Source

The dialysate supply source used for a given dialysis session is preferably one or a set of feeder bags of greater volume, for example 5 liters, than that, for example 150 ml, of said ventricle bag 50, 50′.

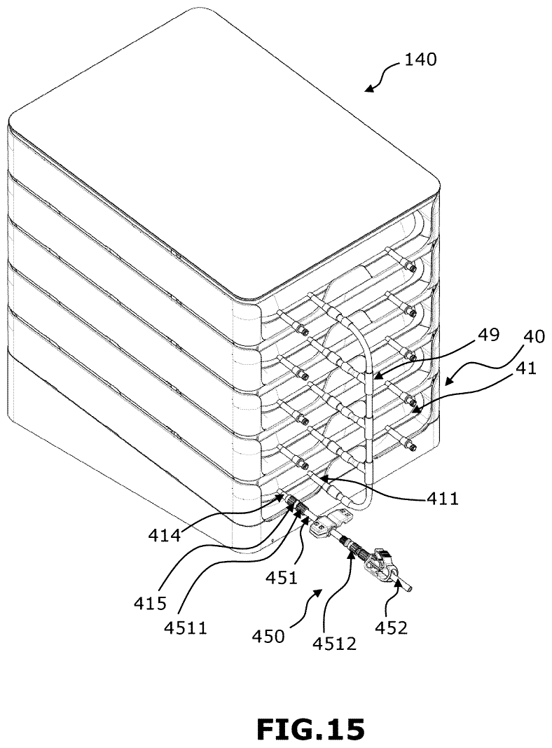

According to one embodiment, and as is illustrated in FIG. 15, each supply source 40, 40′ comprises a plurality of bags 41, called feeder bags, and a connection device 49, called an interconnection octopus, which comprises a plurality of conduits for connecting the feeder bags to each other at the level of endpieces 411, preferably by means of quick-release couplings, for example Luer Lock couplings. According to one embodiment, each dialysate supply source 40 or 40′ can be housed in a support structure 140 with stages. An example of a support structure with stages is described in the international application WO2014001680. Thus, as is illustrated in FIG. 15, each feeder bag 41 of the supply source is positioned in a stage of the support structure 140.

The supply source 40 comprises a conduit 414 provided with a connector (or coupling) 415 which can be connected to and disconnected from the first section 451. The supply source 40′ comprises a conduit 414′ provided with a connector 415′ (preferably identical or similar to the connector 415) which, in the withdrawn state of the first section 451, can be connected to and disconnected from the second section 452 of the connection system 450 presented below. Preferably, the conduit 414 is situated at the level of the lowest feeder bag 41 in the state with the feeder bags positioned in the support structure 140. With a view to a first session, the connector 415 of the supply source 40 is connected to the first section 451. Preferably, the connector 415 cannot be detached; for example it is soldered to the conduit 414 of the supply source 40. The withdrawal of the first section 451 from the second section 452 makes it possible to connect the connector 415′ (of the same type as the connector 415) of the supply source 40′ to the second section 452. Said connector 415′ is also disconnectable from the second section 452 and is preferably fixed in a non-detachable manner to the conduit 414′ of the supply source 40′.

### Supply Source Connection System

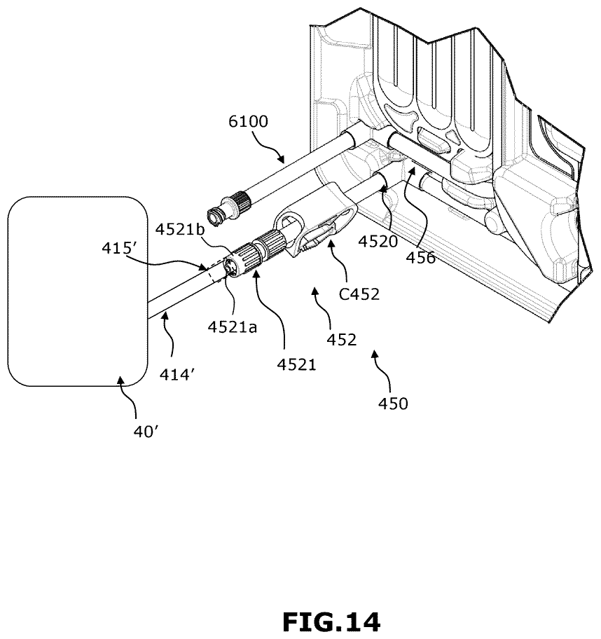

The machine comprises a connection system 450 between a dialysate supply source 40 and the dialysate feed line 52, in particular at the level of a connector 456 (FIG. 14) which communicates with the inlet 511 of the dialysate delivery system 51, in order to provide dialysate to the bags of the dialysate delivery system 51. In the example illustrated in FIGS. 2 and 4, the dialysate supply source used for a first dialysis session is labeled 40, and the one distinct from the supply source 40 and used for a second dialysis session is labeled 40′.

The dialysate supply source used for a given dialysis session is preferably one or a set of feeder bags of greater volume, for example 5 liters, than that, for example 150 ml, of said ventricle bag 50, 50′.

As is illustrated in FIGS. 2 to 4, 7, 9, 11 and 14, the connection system 450 comprises a first section 451 which comprises a conduit 4510 having one end capable of being connected by a connector 4511, preferably a connector of the Luer Lock type, for example a male connector, to a corresponding connector 415, for example a female connector, with which a conduit 414 of the first dialysate supply source 40 is provided. The Luer Lock type connector corresponds to the NF EN ISO 594 standard.

The connector 4511 comprises:
a tubular element 4511a capable of being engaged in an opening of the corresponding coupling (connector) of the dialysate supply source in order to allow liquid communication inside said tubular element; and
a peripheral wall 4511b which extends around and at a distance from the tubular element 4511a in order to define an annular space for insertion of a peripheral wall of said connector of the dialysate supply source.

The conduit 4510 of the first section 451 comprises, at the other end, a connector 4512 configured to be connected to a connector of a second section 452 of the connection system 450.

According to one embodiment, and as is illustrated in FIG. 9, the first section 451 is provided with a clamp system C451, which is initially in the open state and which can be actuated so as to close on the conduit 4510, damaging the conduit 4510 and/or being locked in the closed position. The system C451 is also called a tamper-evident closure member. The use of such a tamper-evident closure member C451 makes it possible to ensure that the first section 451, in particular the corresponding conduit 4510, is not reused.

The connection system 450 also comprises a second section 452, which comprises a conduit 4520 having one end capable of being connected to a second dialysate supply source 40′.

For this purpose, said end of the second section 452 is provided with a connector 4521, for example of the male Luer Lock type.

The connector 4521 comprises:
a tubular element 4521a capable of being engaged in a second connector opening 4512 of the first section 451 in order to allow liquid communication inside said tubular element; and
a peripheral wall 4521b which extends around and at a distance from the tubular element 4521a in order to define an annular space for insertion of the peripheral wall of said connector of the dialysate supply source.

Preferably, the other end of the second section 452 is connected to a connector 456 which is in fluidic communication with the inlet 511 of the delivery system 51.

According to a preferred embodiment, the second section 452 is equipped with a clamp system C452 for opening/closing the second section 452.

For a first dialysis session, and as is illustrated in FIG. 9, the dialysate supply source 40 is connected to the first section 451 of the connection system 450 by its conduit 414 provided with a connector adapted to be connected to the connector 4511 of the first section 451. The connector of the conduit 414 is preferably a female Luer Lock connector, for example a connector of the type designated 4512, 821 or 921.

During this first dialysis session, the clamp system C451 is left in the open state, likewise the clamp system C452, in order to allow the dialysate coming from said supply source to supply the delivery system 51 which is connected to the feed line 52. The dialysate can thus flow from the supply source 40 to the delivery system 51 by passing through the first section 451 and the second section 452.

Advantageously, the conduits 4510, 4520 of the connection system 450 are in the form of flexible tubes, so that they can be pinched. The conduits 4510, 4520 have, in the non-pinched state (or in the non-crushed state), a cross section of constant (external and internal) diameter over their entire length. In other words, the conduits 4510, 4520 do not have intermediate reserves or pockets (for example of liquid) between their ends.

The supply inlet 511 of the dialysate delivery system 51 can correspond to a node linking the inlets of the bags 50, 50′.

After the first given dialysis session, the system C451 is closed by the operator (the user or a third party) in order to render the first section 451 unusable, and the first section 451 is disconnected from the second section 452 (disconnection of the connector 4512 from the connector 4521) in order to be discarded.

Then, for a second given dialysis session, the second section 452 is connected to another dialysate supply source 40′ whose features may be similar or identical to those of the dialysate supply source 40.

In other words, the supply source 40 is withdrawn with the section 452 to which the supply source 40 was connected, and the supply source 40′ is connected to the following section 451 of the connection system 450.

It will be understood that the connection system 450 can have a greater number of successive sections so as to make it possible to carry out as many dialysis sessions as there are sections, by removing the section used after each dialysis session, so as to be able to use the following section for the next dialysis session. For this purpose, provision can be made for the second section itself to be formed of a plurality of sections (sub-sections) connected in series.

It will be understood that the bags 50, 50′ are fed by means of the dialysate supply source 40 or 40′ which is outside the enclosures, in particular outside the cassette 9.

As has been set out above and as is illustrated in FIG. 15, each supply source 40 or 40′ comprises a plurality of bags 41, called feeder bags.

As has been set out above, each section 451 or 452 of the connection system 450 can thus be selectively connected to a conduit 414 of the lowest feeder bag 41 in the state with the feeder bags positioned in the support structure 140.

Such a combined use of the connection system 450 and of the dialysate delivery system 51 with flexible bag avoids the need to open the enclosures 500, 500′, and therefore access the cassette, not only during a dialysis session, but also between two successive sessions, as long as there is still an unused section of the connection system. Indeed, during a session with the supply source 40 and then during the following session with the supply source 40′, the cassette and thus the ventricle bags can be left in the machine without having to be replaced.

### Cassette

As is illustrated in FIG. 7, part of the machine is in the form of a cassette 9 having a body 900. The cassette 9 is a cassette removable from the frame of the machine and is intended to be inserted into a housing of said frame. Said cassette 9 comprises in particular said bag or bags 50, 50′, a body 900, and the feed line 52. According to a particular aspect, the cassette 9 also comprises the dialysate discharge line 62 and the discharge bag or bags 60, 60′ of the discharge system.

In the example illustrated in the figures, said cassette 9 comprises in particular the dialysate lines 52, 62 and each bag 50, 50′, 60, 60′. The cassette can comprise the system 450 for connecting the dialysate supply source 40, 40′ to the delivery system 51 and/or the system 800, 900 for connecting to the dialyzer.

According to one embodiment, the pressure sensors 523, 524, 623, 624, shown in FIGS. 2 to 4, are fixed to the frame of the machine. When the cassette 9 is inserted into the machine, the pressure measurement conduits are in pneumatic communication with the pressure sensors. Pneumatic communication signifies that the sensor and the corresponding pressure measurement conduit are arranged in relation to each other in such a way that the sensor is able to measure the pressure prevailing in said conduit, and therefore the pressure prevailing in the line at the point where the corresponding pressure tap orifice is provided.

### Control Unit

As has been mentioned above, said machine also comprises a control unit 10, such as a programmable logic controller. The control unit can be configured to execute dialysis steps, as are described in the international applications WO2013050689A1 or WO2019150058A2.

In addition to the initiation and return phases, a dialysis session also comprises a dialysis phase, which can comprise a plurality of ultrafiltration steps in alternation with backfiltration steps. An example of the operation of the machine during a dialysis session with a plurality of ultrafiltration steps in alternation with backfiltration steps is described in the international application WO2013050689 A1 or WO2019150058 A2. However, it should be noted that the dialysis phase generally comprises an exchange phase between the blood compartment and the dialysate compartment, for example by osmosis, and may not include a backfiltration phase and/or ultrafiltration phase.

For the dialysis phase, the control unit 10 controls in particular the means for pressurizing the bags 50, 50′ and the clamp systems C1, C2; C1′, C2′ and C5, C6; C5′, C6′ for closing/opening the feed system 5 and the discharge system 6. In a dialysis session, the initiation phase corresponds to the arrival of the blood in the blood compartment of the dialyzer. For this initiation phase, the control unit controls the blood pump Psg.

The dialysis session ends with a return phase during which the blood present in the blood circuit (venous line, arterial line, and blood compartment of the patient) is returned to the patient. For this return phase, the control unit also controls the blood pump Psg, a bag of physiological saline being connected to the line L1 in order to allow the blood to be returned without the risk of air being introduced.

According to one embodiment, the control unit 10 is further configured to implement a method for rinsing and eliminating air from the extracorporeal circuit of the dialysis machine, for example by carrying out rinsing and/or air removal steps, as are described in the application FR 2001604, filed on Feb. 18, 2020 and not yet published.

Said control unit 10 is in the form of an electronic and computer system which comprises, for example, a processor, such as a microprocessor, a working memory and a data memory. Said control unit can be in the form of a microcontroller.

In other words, the functions and steps described can be implemented in the form of a computer program or via hardware components (e.g. programmable gate arrays). In particular, the functions and steps performed by the control unit or by modules of the latter can be performed by sets of instructions or computer modules stored in a memory for their execution by a processor or controller or can be performed by dedicated electronic components or FPGA or ASIC type components. It is also possible to combine computer parts and electronic parts.

When it is specified that the unit or means or modules of said unit are configured to perform a given operation, this signifies that the unit comprises computer instructions and the corresponding means of execution to allow said operation to be performed and/or that the unit comprises corresponding electronic components.

Said control unit also has a data input interface. Provision can be made that said interface allows input of data relating to the patient and/or to the consumables of the machine.

### Other Components or Means

In the example illustrated in the figures, the dialysis machine is also provided with conventional components to ensure reliable and efficient treatment of the body fluid to be treated, in particular a blood leak detector FS provided in the discharge line 62. Advantageously, the control unit 10 is configured to allow the flowmeters to be calibrated with respect to one another.

Each enclosure 500, 500′ is provided with means for heating or preheating the enclosure. Each enclosure 500, 500′, 600, 600′ is also provided with a vent valve V2, V5, V8, V11 associated with an air filter F1, F2, F3, F4, and with a pressure sensor P7, P8, P9 and P10.

The pressurizing means 70 comprise the following elements: a compressed air tank R1, a pressure sensor P11 for the tank R1, a compressor Pa1, a non-return valve for the compressed air Ar1, and a compressed air filter and silencer S.

The vacuum generator 80 comprises an air vacuum tank R2, a pressure sensor P12 for the tank R2, a vacuum pump Pa2, a non-return valve for the air vacuum Ar2, and an air filter and silencer Si2 for the air vacuum.

The feed line 52 is also provided with means Ch3 for heating the dialysate, associated with means for measuring temperature T1, T2, T3. The discharge line 62 is provided with means for measuring temperature T4, T5 downstream of the bags 60, 60′.

Advantageously, the venous line L2 also comprises a closing/opening member CV, such as a clamp. The machine can comprise a bypass line L10, and also one or more associated closing/opening members Vbp. The bypass line L10 can be used to perform the initiation and calibration of the dialysate circuit, and to make it possible to avoid injecting defective dialysate into the dialyzer, for example due to a temperature problem.

Advantageously, the discharge line of the dialyzer is connected to a recovery container or to a drainage system Egt.

### Method

According to one embodiment, the implementation of a dialysis session with a dialysis machine as described above comprises the steps described below. As has been mentioned above, when technically permissible, the order of certain steps can be reversed and the method can comprise, between two steps described, other additional steps. The method described below permits prolonged use of a cassette after the completion of a first dialysis session, for the execution of a second dialysis session with the same cassette, with a reduced risk of contamination during the intervention of the operator for changing the dialyzer and/or for changing the dialysate supply source.

With a view to a first use of a cassette 9, in step 1600, the user opens a door of the dialysis machine in order to insert the cassette 9 into a corresponding housing (for example visible in FIG. 7).

The presence of the stoppers 829 and 929 makes it possible to limit the risk of contamination of the connectors 820 and 920 when the cassette is taken out of its packaging for the first session and before it is mounted in the machine and before connection to the dialyzer.

As is shown in FIGS. 2 and 8, the user opens the stoppers 829 and 929 and connects the feed line 52 to the inlet 401 of the dialysate passage zone 14 of the dialyzer 100 with the connection system 800 and connects the discharge line 62 to the outlet 402 of the dialysate passage zone 14 of the dialyzer 100 with the connection system 900 (step 1610).

The clamp systems C451, C452 are in the open position, so that the conduits 451, 452 are not pinched.

The user places feeder bags 41 of dialysate from the supply source 40 in the support structure 140. The user connects the feeder bags 41 to each other with the aid of the interconnection octopus 49.

The user installs and connects a bag of physiological saline on the line L1 upstream of the pump Psg.

The user connects the venous line L2 and arterial line L1 of the extracorporeal circuit to the dialyzer 100.

With a view to a first dialysis session, and as is illustrated in FIGS. 2 and 9, the user connects, if necessary, the first section 451 of the connection system 450 to the supply source 40 (step 1620). According to a preferred embodiment, the connection system 450 is provided (delivered) in a configuration in which the first section 451 and the second section 452 are already connected to each other, the second section 452 also being connected to the delivery system 51. The clamp system C452 of the second section is left open to allow the dialysate from the supply source 40 to supply the delivery system 51 via the sections 451 and 452 of the connection system. Note that the clamp system C451 is also left open for as long as the first dialysis session is not finished.

Thus, for this first dialysis session, the connector 4511 of the first section 451 is connected to the connector 415 of the first dialysate supply source 40, while the second section 452 is in fluidic communication with the first section 451 via the connectors 4512, 4521. The clamp systems C451, C452 are in the open position.

The user switches on the machine in order to start the machine running. The control unit acquires, for example by input in a data interface and/or by reading in a memory, data relating to the consumables or to their batch, such as data identifying the dialysate supply source, the dialyzer, the extracorporeal circuit, and the cassette.

The user can perform settings using the input interface, including entering the “dry” and actual weight data in order to allow the control unit to determine the amount of excess aqueous fraction to be removed from the body fluid.

When the conditions for executing a dialysis session are fulfilled, as has been explained above, the control unit can command the execution of a first dialysis session comprising sequences of initiation, dialysis and return. The initiation sequence can be preceded by a step of rinsing the consumables by circulating dialysate in the dialysate circuit, in particular in the cassette. In parallel, physiological saline is circulated in the extracorporeal circuit (blood lines and blood compartment) in order to rinse this extracorporeal circuit. The patient can then connect to the line L1.

At the end of the first session, the control unit controls the purging of the dialysate present in the delivery system 5 and the discharge system 6.

The machine can then be put into standby, potentially intermittently.

After the first dialysis session, the user removes certain consumable elements from the machine, such as arterial line L1 and venous line L2, and the dialyzer. The cassette 9 remains in the machine, with the exception of the connectors 820 and 920 which the operator disconnects from the feed line 52 and the discharge line 62 with respect to the dialyzer 100, as is illustrated in FIG. 10, in order to replace them with new connectors 820′ and 920′ which are connected respectively to the connectors 810 and 910, as is illustrated in FIGS. 3, 4 and 12, the stoppers 829′ and 929′ being initially in the closed position.

The user also removes the first supply source 40 (step 1630). Preferably, the tamper-evident clamp system C451 is closed after the first session, before or after removal of the supply source.

Preferably, that end of the connection system formed by the connector 4511 is closed by a stopper, which may be a stopper 4519 initially present on this connector 4511 and which has previously been removed in order to be able to connect the first supply source 40, before proceeding to the first dialysis session.

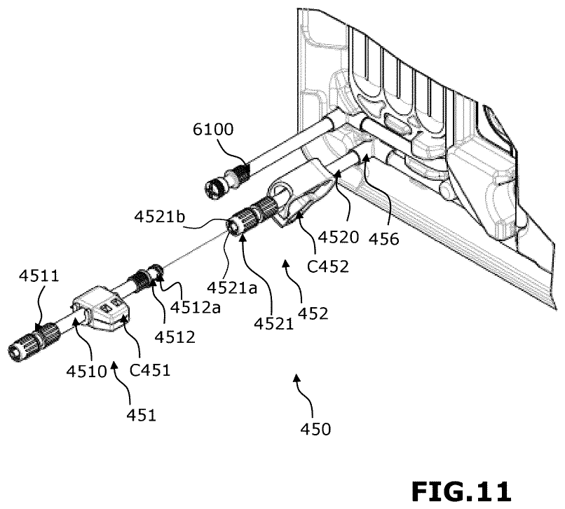

For the implementation of a second dialysis session, in step 1640, the first section 451 of the connection system 450 is removed, as is illustrated in FIG. 11, which leaves the connector 4521 of the second section 452 free so that the connector 415′ of another supply source 40′ can be connected thereto.

The first section 451 is preferably removed just before the second section 452 of the connection system 450 is connected to the second dialysate supply source 40′.

Thus, for the implementation of a second dialysis session, and as is illustrated in FIG. 14, the second section 452 of the connection system 450 is connected to the second dialysate supply source 40′ (step 1650). According to one embodiment, in the same way as for the supply source 40, the feeder bags of the supply source 40′ are interconnected with the aid of an interconnection octopus. The user opens the clamp system C452 (which may have been previously closed after the first dialysis session) in order to free the fluidic communication inside the second branch 452.

A new venous line L2′ and arterial line L1′ are connected to a new dialyzer 100′. The user installs and connects a new bag of physiological saline.

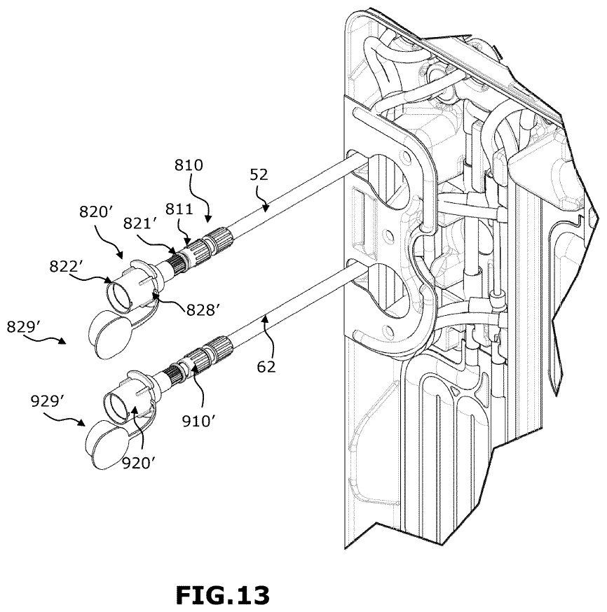

The stoppers 829′, 929′ of the new connectors 820′, 920′ are then opened, as is illustrated in FIG. 13, in order to connect the feed line 52 to the inlet 401 of the dialysate passage zone 14 of a new dialyzer 100′ and the discharge line 62 to the outlet 402 of the dialysate passage zone 14 of the new dialyzer 100′.

As with the first session, the user can enter parameters with the aid of the input interface for the second dialysis session.

When the conditions for executing a new dialysis session are fulfilled, as has been explained above, the control unit 10 can command the execution of a second dialysis session comprising sequences of initiation, dialysis and return.

The stoppers 829′ and 929′ on the parts 820′ and 920′, available for the second session, protect the latter from contamination when they are taken out of their unit packaging before the start of the second session.

The invention is not limited to the embodiments illustrated in the drawings.

Furthermore, the term “comprising” does not exclude other elements or steps. In addition, features or steps that have been described with reference to one of the embodiments explained above may also be used in combination with other features or steps of other embodiments explained above.

1. A dialysis machine for treating a body fluid, such as blood or plasma, the dialysis machine comprising:
a dialyzer comprising an enclosure which includes a body fluid passage zone which has a body fluid inlet and a body fluid outlet, and a dialysate passage zone which has a dialysate inlet and a dialysate outlet, and a membrane system between the body fluid passage zone and the dialysate passage zone;
a dialysate delivery system,
a connection system for connecting a dialysate supply source to the dialysate delivery system, the connection system comprising:
a first section, which comprises a conduit provided at one end with a first connection device which is connectable to a connector of a first dialysate supply source, and which is disconnectable from said connector of the first dialysate supply source, and provided at the other end with a second connection device, said conduit being a flexible conduit of constant cross section in the uncrushed state of said conduit, and
a second section, which comprises a conduit which has one end connected to a dialysate delivery system and which is provided at an opposite end with a connection device which is connected, in a disconnectable manner, to the second connection device of the first section, in order to provide a series connection of the first section and the second section, while allowing said first section to be disconnected from the second section, said conduit of the second section being a flexible conduit of constant cross section in the uncrushed state of said conduit,
so that, in the state with the first section disconnected and removed from the second section, said connection device of the second section is connectable to a connector of a second dialysate supply source and disconnectable from said connector of the second dialysate supply source.

2. The machine as claimed in claim 1, wherein the connection system comprises a closing element which, in the closed state, makes it possible to prevent liquid from circulating inside the conduit of the first section.

3. The machine as claimed in claim 2, wherein the closing element is of the tamper-evident type so that, in the closed state, the closing element is locked and/or damages the conduit of the first section in order to prevent reuse of the conduit after the closing element has been closed.

4. The machine as claimed in claim 1, wherein the second section comprises an activatable and deactivatable opening and closing element making it possible to allow or prevent the circulation of liquid through the conduit of the second section.

5. The machine as claimed in claim 1, wherein the first connection device of the first section comprises:
a tubular element capable of being engaged in an opening of the corresponding connector of the first dialysate supply source in order to allow liquid communication inside said tubular element; and
a peripheral wall which extends around and at a distance from the tubular element in order to define an annular space for insertion of a peripheral wall of said connector of the first dialysate supply source.

6. The machine as claimed in claim 1, wherein the connection device of the second section comprises:
a tubular element which is capable of being engaged in an opening of the second connection device of the first section in order to allow liquid communication inside said tubular element, and which, in the withdrawn state of the first section, is capable of being engaged in an opening of the connector of the second dialysate supply source in order to allow liquid communication inside said tubular member; and
a peripheral wall which extends around and at a distance from the tubular element in order to define an annular space for insertion of a peripheral wall of said second connection device of the first section or, in the withdrawn state of the first section, to define an annular space for insertion of a peripheral wall of the connector of the second dialysate supply source.

7. The machine as claimed in claim 1, wherein the second connection device of the first section comprises a peripheral wall provided with a thread able to cooperate with an internal thread formed in the peripheral wall of the connection device of the second section.

8. The machine as claimed in claim 1, wherein the machine comprises:
a dialysate feed line which is connectable to an inlet of a dialysate passage zone of the dialyzer, and
a dialysate discharge line which is connectable to an outlet of the dialysate passage zone of the dialyzer.

9. The machine as claimed in claim 8, wherein the discharge line comprises a conduit for connection to a discharge device such as a drainage system and/or a spent dialysate recovery system.

10. The machine as claimed in claim 1, wherein the machine comprises at least one flexible bag, called a ventricle bag, intended to contain dialysate and forming part of said delivery system.

11. The machine as claimed in claim 10, wherein said at least one bag is included in a device, called a cassette, which is insertable into and removable from a corresponding housing provided in the dialysis machine.

12. The dialysis machine as claimed in claim 11, wherein the machine comprises a frame and said device, called a cassette, is removable from the frame of the machine.

13. A method for successively connecting dialysate supply sources to a connection system of the machine as claimed in claim 1, the method comprising the following steps:
providing the connection system in a configuration in which the first section is connected to the second section, the second section being connected by its other end to the dialysate delivery system;
providing a first dialysate supply source having a connector not connected to the connection system;
when a stopper closes the end of the first section opposite the end connected to the second section, removing said stopper;
connecting the first connection device of the first section to the first dialysate supply source, and, after use of the first dialysate supply source:
preferably closing off the first section, for example by closure of a clamp system on the first section,
preferably removing the first dialysate supply source and closing the connection device of the first section with the aid of a stopper;
removing the first section from the second section of the device so that the second section has a free connection device;
providing a second dialysate supply source having a connector not connected to the connection system;
connecting the connector of the second dialysate supply source to the free connection device of said second section.
